(19) United States
(12) Patent Application Publication
(10) Pub. No.: US 2019/0184088 A1
(43) Pub. Date: Jun. 20, 2019

(54) Nasal Irrigation Apparatus and Kit

(71) Applicant: Rhinoclear Nasal Care Solutions Inc., Calgary (CA)

(72) Inventor: Bradford Mechor, Calgary (CA)

(21) Appl. No.: 16/326,045

(22) PCT Filed: Aug. 15, 2017
(86) PCT No.: PCT/CA2017/050967

§ 371 Date: Feb. 15, 2019

Related U.S. Application Data
(60) Provisional application No. 62/506,863, filed on May 16, 2017.
(60) Provisional application No. 62/375,237, filed on Aug. 15, 2016.

(51) Int. Cl. A61M 3/02; A61H 35/04; A61M 11/00

(52) U.S. Cl. CPC A61M 3/0262; A61H 35/04; A61M 15/08; A61M 3/0279; A61M 11/008

(57) ABSTRACT

An apparatus for nasal irrigation comprising a resiliently compressible bottle component and a spout having a proximal end for demountable engagement with the bottle component. The distal end of the spout may be molded for sealable engagement with a user's nostril. Alternatively, the distal end of the spout may be threaded for demountable sealable engagement with a nasal tip molded for sealable engagement with a user's nostril. The tip may be configured for delivering a solution in the form of a stream, or a spray, or a misting. A kit comprising the nasal irrigation apparatus and one or more selected volumes of one or more nasal irrigation solutions. The kit may additionally comprise one or more nasal tips. The nasal irrigation solution may comprise one or more of an antibiotic, an antihistamine, a steroid, a vasoconstrictor, an emollient, and an ion.

### TECHNICAL FIELD

This disclosure relates to nasal irrigation. More specifically, this disclosure pertains to apparatus and solutions delivered thereby for nasal irrigation.

### BACKGROUND

Air enters the human lungs through two primary routes, i.e., the nose and the mouth. Air enters the nose through two nostrils which are passages into the nasal cavity from which the air flows into the nasopharynx, then the laryngopharenx, then the larynx and through the trachea into the lungs.

Air is conditioned by filtering through hairs in the nostril and then humidified and warmed in the nasal cavity. The entire surface of the nasal cavity is generally covered with a blanket (i.e., layer) of mucus which entraps particulate matter removed from the air. During good health conditions, the mucus layer is slowly moved by ciliated columnar epithelial cells that line the nasal cavity, toward the pharynx where it passes into the esophagus and moved to the stomach where it is digested.

The nasal cavity comprises four paired paranasal sinuses i.e., (i) the maxillary sinuses which are directly connected to the nasal cavity and which separate and space from the nasal cavity, (ii) the ethmoidal sinuses, (iii) the sphenoidal sinuses, and (iv) the frontal sinuses. The paranasal sinuses are also covered with blankets of mucus that are moved slowly toward the pharynx and on into the stomach.

All individuals experience periods of nasal congestion during which their ability to breathe through their noses is significantly impaired due to excessive production and accumulation of mucal secretions and phlegm in the nasal cavity, the maxillary sinuses, and the pharynx. Congestion commonly occurs as a consequence of one or more of a viral infection of the upper respiratory tract, a bacterial infection of the upper respiratory tract, or an allergic reaction. Congestion is often accompanied by considerable physical discomfort associated with one or more of post-nasal drip, decreased sense of smell, ear aches, headaches, sore throat, malaise, muscle and joint aches, fatigue, cough, chest congestion, fever, chills and gastrointestinal maladies.

Among treatments used to lessen the discomfort associated with congestion is irrigation of the nasal cavity and the maxillary sinuses with a solution to flush away the excessive accumulations of mucal secretions and phlegm. There are many types of containers that have been used for delivery of nasal irrigation solutions. For example, resiliently squeezable cylindrical bottles and bulb-shaped bottles, and the like. Such resiliently squeezable bottles are typically connected to a spout having a tip at its distal end to insert into and plug a nostril opening so that the irrigation solution is forced into the nasal cavity with the bottle is squeezed. Other types of containers are those referred to as “teapot” containers having spouts with nasal tips for delivering irrigation solutions into users' nostrils when their heads are tipped back with the teapots held above their noses. Other types of container are provided with user-controlled hand-pumps to deliver irrigation solutions into their nostrils.

However, there are many problems associated with the apparatus used for delivery of irrigation solutions into users' nostril. For example, resiliently squeezable bottles are difficult for users to handle to comfortably and completely deliver the bottle contents into their nasal cavities. Regardless of the type of container used, the delivery process, in addition to uncomfortable operation, is messy and causes in spillage and dripping of the irrigation solution over the users' faces and their clothes. Furthermore, the containers used for delivery of nasal irrigation solutions require the user to tilt their head to one side or alternatively, to tilt their head back while self-administering the flow of irrigation solution into one nostril, further adding to the discomfort of and dissatisfaction with using such apparatus. Yet another problem is the necessity to clean and sterilize the containers and their spouts after each use so that bacterial and/or viral infections present in lavaged mucal secretions and phlegm are not re-introduced into the users' naval cavities.

### SUMMARY

The embodiments of the present disclosure relate to an apparatus for nasal irrigation comprising a resiliently compressible bottle component, a spout having a proximal end for demountable engagement with the bottle component and a distal end for demountable engagement with a nasal tip, and at least one nasal tip for delivering a solution in the form of a stream, or a spray, or a misting.

Some embodiments of the present disclosure relate to a kit comprising the nasal irrigation apparatus, and one or more selected volume(s) of a nasal irrigation solution.

### BRIEF DESCRIPTION OF THE FIGURES

The present disclosure will be described in conjunction with reference to the following drawings in which:

FIG. 1 is a side view of a nasal irrigation apparatus according to one embodiment of the present disclosure;

FIG. 2 is a front view of the nasal irrigation apparatus shown in FIG. 1;

FIG. 3 is a cross-sectional side view of the nasal irrigation apparatus shown in FIGS. 1 and 2;

FIG. 4 is a side view of the bottle component of the nasal irrigation apparatus shown in FIGS. 1 and 2;

FIG. 5A is a side view of the cap component of the nasal irrigation apparatus shown in FIGS. 1 and 2, FIG. 5B is a front view of the cap component, and FIG. 5C is a cross-sectional side view of the cap component;

FIG. 6A is a side view of another embodiment of a cap component for a nasal irrigation apparatus disclosed herein, and FIG. 6B is a cross-sectional side view of the cap component shown in FIG. 6A;

FIG. 7A is a top view of the tip component of the nasal irrigation apparatus shown in FIGS. 1 and 2, FIG. 7B is a side view of the tip component, and FIG. 7C is a cross-sectional side view of the tip component;

FIG. 8A is a top view of another embodiment of a tip component for a nasal irrigation apparatus disclosed herein, and FIG. 8B is a close-up top view of the tip component shown in FIG. 8A;

FIG. 9A is a top view of another embodiment of a tip component for a nasal irrigation apparatus disclosed herein, and FIG. 9B is a close-up top view of the tip component shown in FIG. 9A;

FIG. 10 is a side view of a nasal irrigation apparatus according to another embodiment of the present disclosure;

FIG. 11 is a front view of the nasal irrigation apparatus shown in FIG. 10;

and

FIG. 12 is an exploded cross-sectional side view of the nasal irrigation apparatus shown in FIGS. 10 and 11.

### DETAILED DESCRIPTION

The embodiments of the present disclosure relate to apparatus for self-administered delivery of nasal irrigation solutions by a user. According to one aspect, an exemplary apparatus disclosed herein comprises: (i) a resiliently compressible container with a cylindrical body having at least one plurality of molded finger grip channels extending partially around the container, (ii) a molded spout having a shepherd's hook shape wherein the proximal end of the spout is demountably and sealingly engageable with the container, and (iii) a nasal tip for sealingly engaging a user's nostril, wherein the nasal tip is demountably and sealingly engageable with the distal end of the molded spout. Alternatively, the distal end of the spout may have a molded tip shaped to demountably and sealingly engage a user's nostril.

According to an embodiment, the cylindrical body may have two pluralities of adjacent molded finger grip channels ending partially, or alternatively completely, around the circumference of the resiliently compressible container.

According to another embodiment, the cylindrical body may have three pluralities of adjacent molded finger grip channels ending partially, or alternatively completely, around the circumference of the resiliently compressible container.

According to yet another embodiment, the cylindrical body may have four pluralities of adjacent molded finger grip channels ending partially, or alternatively completely, around the circumference of the resiliently compressible container.

According to an embodiment, the cylindrical body of the resiliently compressible container may have a round shape, alternatively an elliptical shape, or alternatively, an asymmetrical shape.

According to an embodiment, the spout may have a linear form along a single axis without any curvatures.

According to an embodiment, the nasal tip may have a circular orifice at its distal end to provide a stream of irrigation solution into a user's nostril when the resiliently compressible container is squeezed by the user. According to another aspect, the nasal tip may have a rectangular orifice with rounded ends at its distal end to provide a spray of irrigation solution into a user's nostril when the resiliently compressible container is squeezed by the user. According to another aspect, the nasal tip may have a plurality of small-diameter orifices at its distal end to provide a mist of irrigation solution into a user's nostril when the resiliently compressible container is squeezed by the user.

Other embodiments of the present disclosure relate to nasal irrigation solutions compositions formulated to provide a selected type of relief to a user. For example, a nasal irrigation solution may be formulated to provide a cleansing flow to loosen and flush dried phlegm and/or mucus. Alternatively, a nasal irrigation solution may be formulated to deliver one or more emollients for moisturizing and/or soothing irritated mucosal cells. Alternatively, a nasal irrigation solution may be formulated to deliver a medicant, for example an antibiotic, a sleep aid, a motion sickness antidote, an energy and/or alertness aid, and the like.

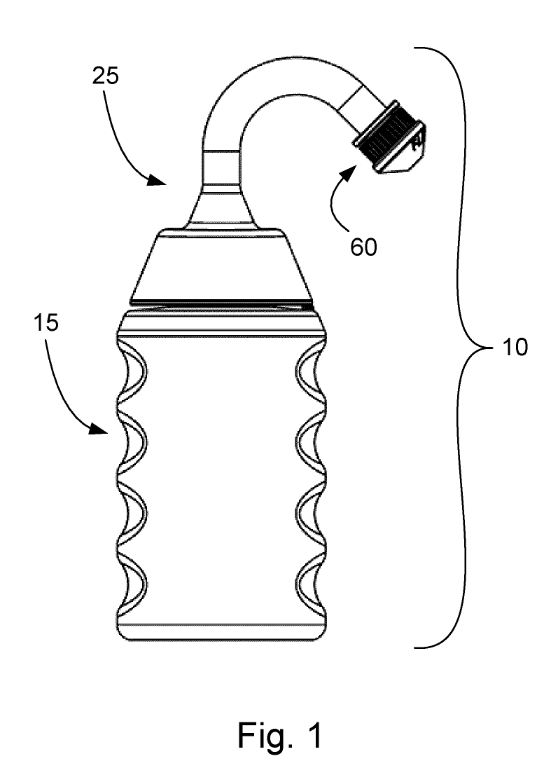

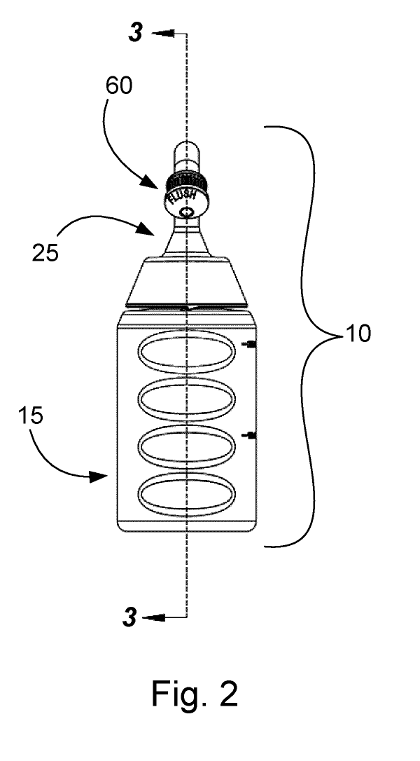

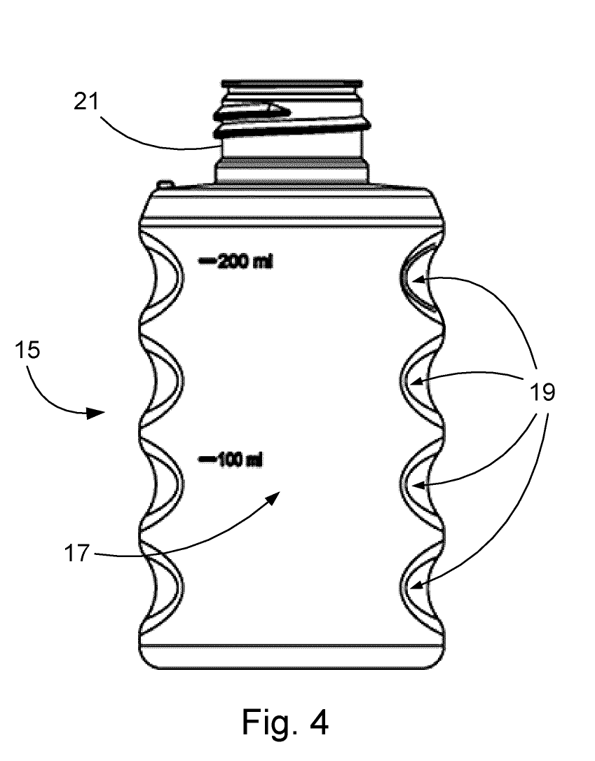

One example of an embodiment of a nasal irrigation apparatus 10 disclosed herein is shown in FIGS. 1-3 and generally comprises a resiliently compressible container 15 to which is demountably and sealingly engaged a spout 25 molded into a shepherd's crook. To the distal end of the spout 25 is demountably and sealingly engaged a nasal tip 60. The resiliently compressible container 15 comprises a body portion 17 and a male-threaded open top 21 (FIG. 4). The resiliently compressible container 15 is provided with four plurality of opposed adjacent channels molded thereinto (FIG. 4) wherein the four pluralities of adjacent channels 19 are spaced equidistantly around the circumference of the container 15. It is to be noted that the four pluralities of opposed adjacent channels 19 will accommodate grasping by a user's thumb and four fingers to facilitate and ease a squeezing action applied to the resiliently compressible container 15. It is suitable for the plurality of adjacent channels 19 to extend around the circumference of the container 15 about 15%, about 20%, about 25%, about 30%, about 40%, about 45% of the diameter of the container. It is optional for the plurality of adjacent channels to extend around the entire circumference of the container 15. It is optional for the resiliently compressible container 15 to be provided with only one plurality of opposed channels 19 molded thereinto (not shown). It is optional to provide a second plurality of opposed adjacent channels (not shown) on container on the side opposite from the first plurality of channels 19. It is optional to provide a third plurality of opposed adjacent channels (not shown) wherein the three pluralities of channels are spaced equidistantly around the circumference of the container 15. It is optional for the plurality of opposed adjacent channels 19 to extend around the container 15 about 25% of the container's diameter, about 30%, about 40% about 50%, about 60%, about 70%, about 80%, about 90%, about 100% of the container's diameter and therebetween.

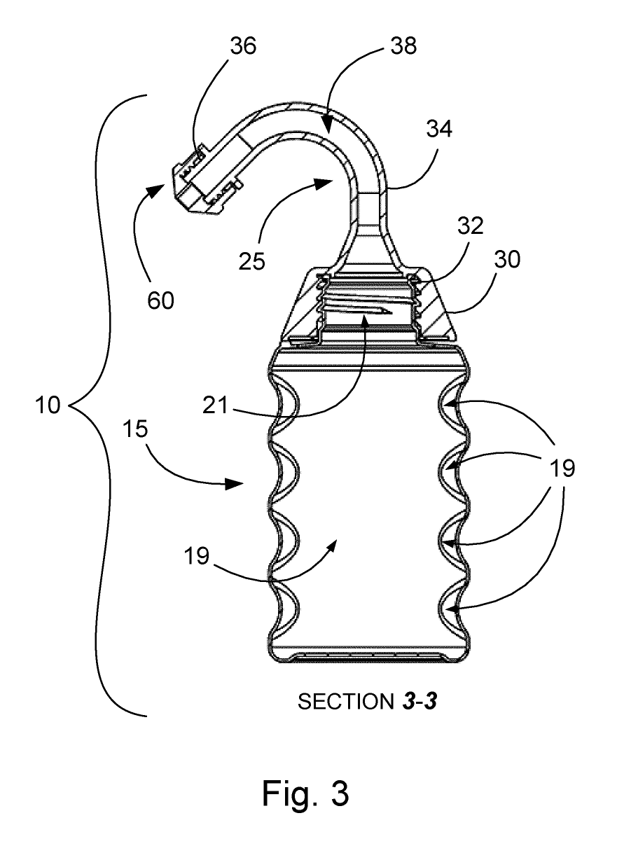

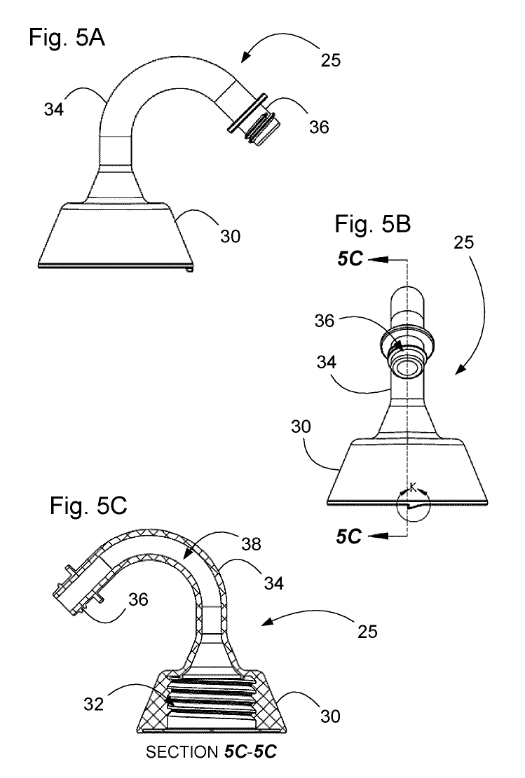

The spout 25 (FIGS. 3, 5A, 5B, 5C) comprises a base 30, a neck 34, and a male-threaded distal end 36 that together define a channel 38 therethrough. The base 30 is provided with a female-threaded orifice 32 (FIG. 5C) for demountable and sealingly engagement with the male-threaded open top 21 of the container 15 (FIG. 4).

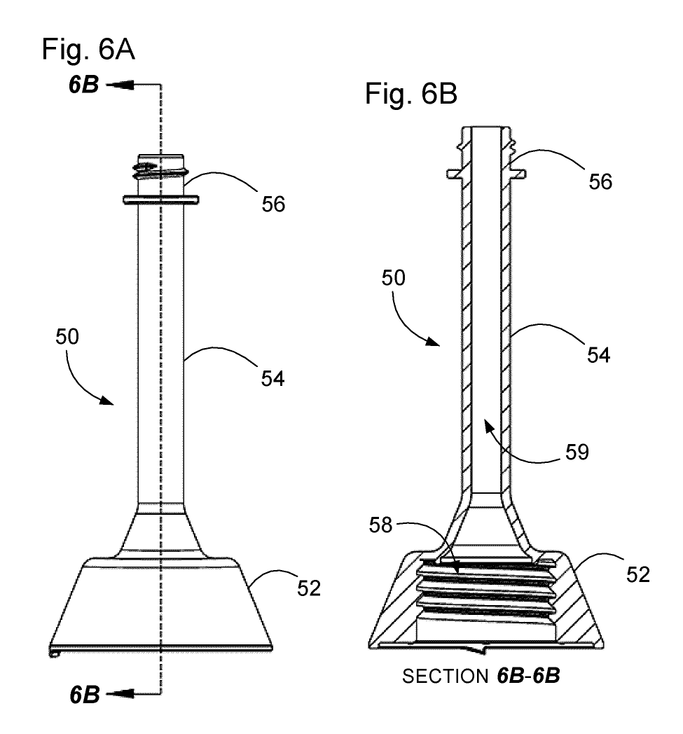

Another suitable spout 50 for demountable sealingly engagement with the male-threaded open top 21 of the container 15 (FIG. 4) is shown in FIGS. 6A, 6B. The spout 50 comprises a base 52 having with a female-threaded orifice 58, an elongate neck 54 extending along a single axis with a male-threaded distal tip 56.

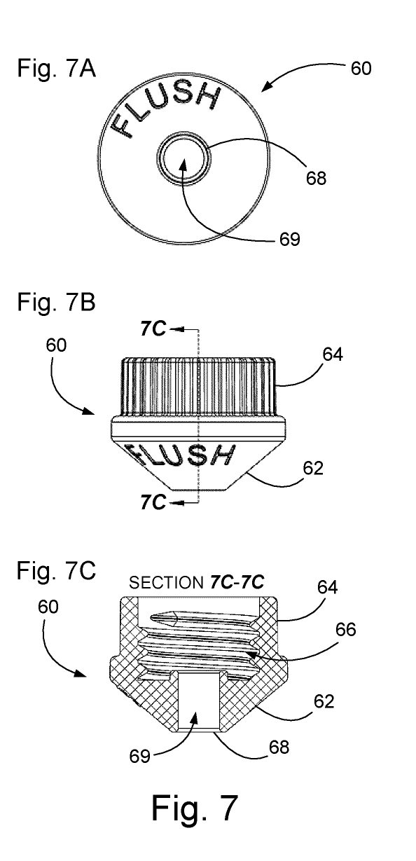

An example of a suitable nasal tip 60 for use with the nasal irrigation apparatus disclosed herein is shown in FIGS. 7A, 7B, 7C. This nasal tip 60 has a cone-shaped distal tip 62 and a proximal base 64 provided a female-threaded orifice 66 (FIG. 7C) for demountable and sealingly engagement with male-threaded distal tip 36 of spout 25 (FIGS. 5A, 5B, 5C) or alternatively, with male-threaded distal tip 56 of spout 50 (FIGS. 6A, 6B). The distal tip 62 is provided with an orifice 69 defined by a circular rim 68 (FIG. 7C). When a user squeezes the resiliently compressible container 15, a strong flow of irrigation solution will be provided through orifice 69 into the user's nostril for dislodging mucal mass and phlegm from the inner surfaces of the nostrils, the nasal cavity, and the maxillary sinuses thereby enabling and facilitating lavage.

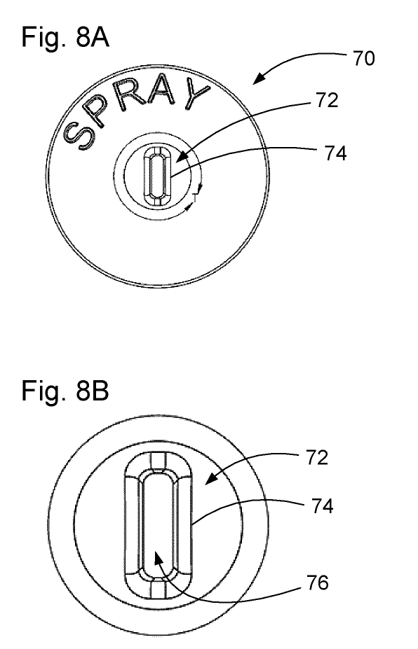

Another example of a suitable nasal tip 70 for use with the nasal irrigation apparatus disclosed herein is shown in FIGS. 8A, 8B. This nasal tip 70 has a cone-shaped distal tip 72 and a proximal base provided a female-threaded orifice (not shown) for demountable and sealingly engagement with male-threaded distal tip 36 of spout 25 (FIGS. 5A, 5B, 5C) or alternatively, with male-threaded distal tip 56 of spout 50 (FIGS. 6A, 6B). The distal tip 72 is provided with rectangular orifice 76 defined by a rectangular rim 74 having rounded ends (FIG. 8B). When a user squeezes the resiliently compressible container 15, a fan-shaped spray of irrigation solution will be provided through orifice 76 into the user's nostril for dislodging and washing mucal mass and phlegm primarily from the inner surfaces of the nostrils and partially into their nasal cavity, thereby enabling and facilitating lavage.

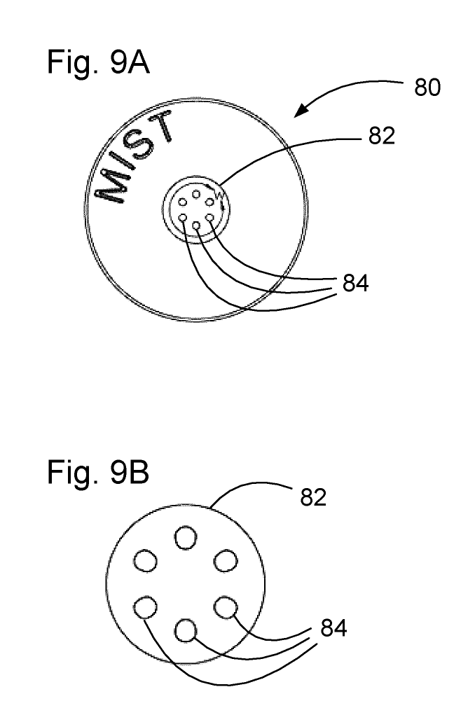

Another example of a suitable nasal tip 80 for use with the nasal irrigation apparatus disclosed herein is shown in FIGS. 9A, 9B. This nasal tip 80 has a cone-shaped distal tip 82 and a proximal base provided a female-threaded orifice (not shown) for demountable and sealingly engagement with male-threaded distal tip 36 of spout 25 (FIGS. 5A, 5B, 5C) or alternatively, with male-threaded distal tip 56 of spout 50 (FIGS. 6A, 6B). The end surface 84 of distal tip 82 is provided with plurality of small-diameter orifices 6 (FIGS. 9A, 9B). When a user squeezes the resiliently compressible container 15, a mist of irrigation solution will be provided through orifices 86 into the user's nostril for providing, for example, one or more emollients and/or moisturizers for soothing irritated and/or inflamed mucosal membranes in the user's nostrils.

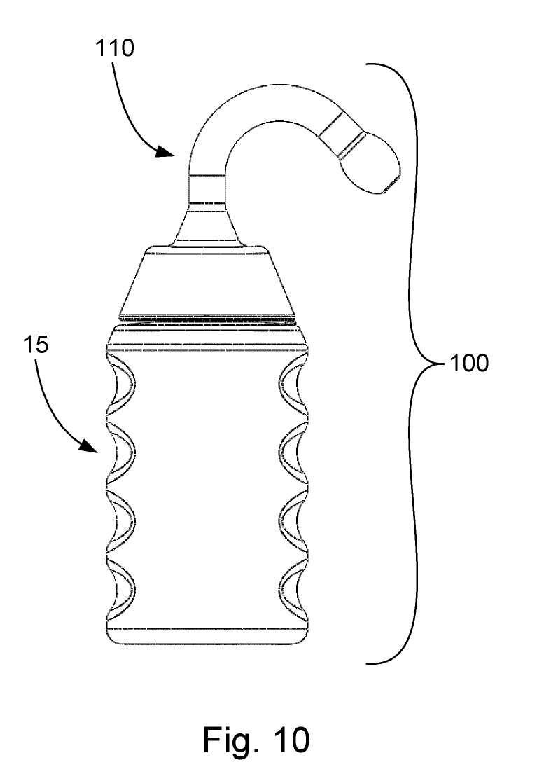

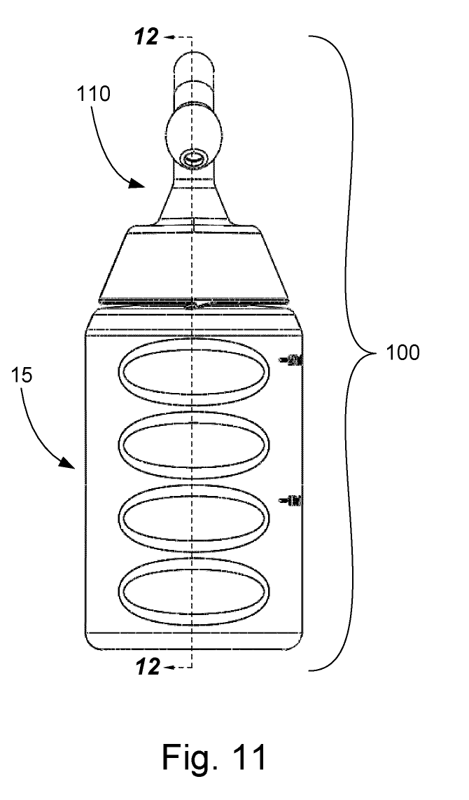

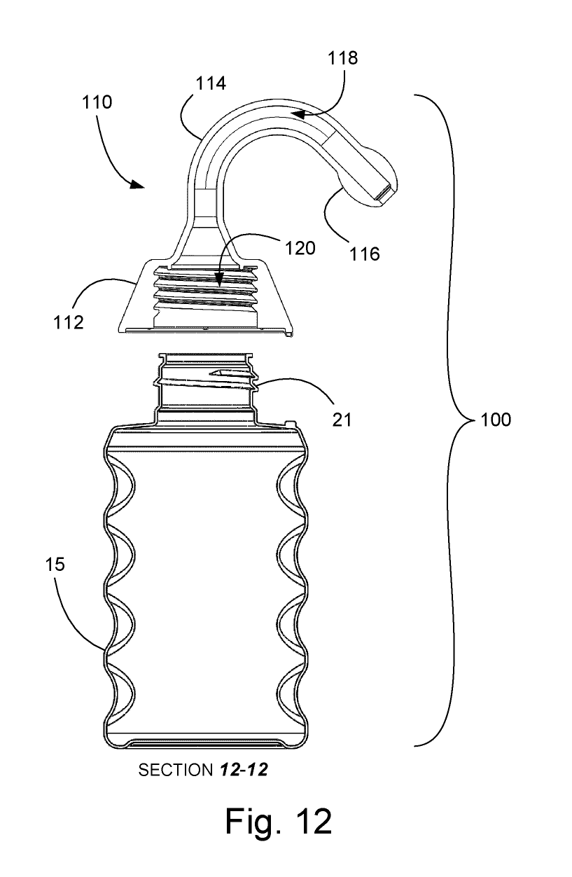

Another example of an embodiment of a nasal irrigation apparatus 100 according to the present disclosure is shown in FIGS. 10-12 and generally comprises the resiliently compressible container 15 shown in FIG. 4, to which is demountably and sealingly engaged a spout 110 molded into a shepherd's crook. In this embodiment, the spout 110 comprises a base 112 at its proximal end, a neck section 114, and a molded tip 116 at its distal end that together, define that together define a channel 118 therethrough. The base 112 of the spout 112 has a female-threaded orifice 120 for demountable and sealingly engagement with the male-threaded open top 21 of the container 15. The tip 116 is molded into a bulbous shape with sufficient girth to sealingly engage a user's nostril. It is to be noted that the tip 116 may be alternatively cone-shaped. It is within the scope of this disclosure for the spout 50 shown in FIGS. 6A, 6B to have an integrally molded bulbous tip at its distal end (not shown).

It is particularly suitable for the container components of the nasal irrigation apparatus disclosed herein, to be sized to provide a single-use dosage volume of a nasal irrigation solution. For example, a suitable volume may be selected from the range of 50 mL to 200 mL, 75 mL to 200 mL, 100 mL to 200 mL, 125 mL to 200 mL, 150 mL to 200 mL, 175 mL to 200 mL, and therebetween.

The shepherd's hook shape of the spout 25 (FIGS. 1-3) and the plurality of plurality of channels 19 molded into the container 15, greatly facilitate the ease of use of the nasal irrigation apparatus disclosed herein. A user would simply turn the container upside down so that the bottom of the container 15 is pointing upward. This upside down orientation of the container will result in a headspace of about 10%, 15%, 20%, 25%, 30%, 35%, 40%, 45%, 50% of the total volume above the level of the irrigation solution in the container. The user would then grasp the container with each of their fingers of the grasping hand sitting in one of the pluralities of channels, then insert and compress the nasal tip against one of their nostrils, then squeeze the resiliently compressible container to force the irrigation solution from the container 15 into their nostril, nasal passage, and maxillary sinuses. After the entire volume of irrigation solution has been expelled, they would then remove the nasal tip 60 (or nasal tip 70 or nasal tip 80 or molded tip 116) from their nostril and complete the lavaging process.

It is suitable for the nasal tip 60 to be made with a soft, pliable and compressible material to enable the user to insert the nasal tip partially into a nostril opening and then to compress the nasal tip against the nostril so that there is no leakage of irrigation solution between the nostril opening and the nasal tip when the user squeezes the resiliently compressible container 15 to force the irrigation solution from the container into the user's nasal passage.

It is within the scope of the present disclosure for the containers, the spouts, and the nasal tips to be made with any suitable resilient polymeric materials. It is suitable to use biobased polymers that may be compostable and/or biodegradable.

Accordingly, one embodiment of a nasal irrigation apparatus disclosed herein may be a single-use apparatus comprising a container with a total volume capacity of 250 mL, a spout, and a nasal tip, all made from one or more biodegradable and/or compostable polymers. According to an aspect, the container may have markings to show one or more of 50 mL, 75 mL, 100 mL, 125 mL, 150 mL, 175 mL, 200 mL, and 250 mL. After use, the apparatus may then be disposed of and recycled.

Another embodiment of a nasal irrigation apparatus disclosed herein may be a single-use apparatus comprising a container with a total volume capacity of 200 mL, a spout, and a nasal tip, all made from one or more biodegradable and/or compostable polymers. According to an aspect, the container may have markings to show one or more of 50 mL, 75 mL, 100 mL, 125 mL, 150 mL, 175 mL, and 200 mL. After use, the apparatus may then be disposed of and recycled.

Another embodiment of a nasal irrigation apparatus disclosed herein may be a single-use apparatus comprising a container with a total volume capacity of 150 mL, a spout, and a nasal tip, all made from one or more biodegradable and/or compostable polymers. According to an aspect, the container may have markings to show one or more of 50 mL, 75 mL, 100 mL, 125 mL, and 150 mL. After use, the apparatus may then be disposed of and recycled.

Another embodiment of a nasal irrigation apparatus disclosed herein may be a single-use apparatus comprising a container with a total volume capacity of 100 mL, a spout, and a nasal tip, all made from one or more biodegradable and/or compostable polymers. According to an aspect, the container may have markings to show one or more of 50 mL, 75 mL, and 100 mL. After use, the apparatus may then be disposed of and recycled.

Another embodiment of a nasal irrigation apparatus disclosed herein may be a single-use apparatus comprising a container with a total volume capacity of 90 mL, a spout, and a nasal tip, all made from one or more biodegradable and/or compostable polymers. According to an aspect, the container may have markings to show one or more of 50 mL, 70 mL, and 90 mL. After use, the apparatus may then be disposed of and recycled.

Another embodiment of a nasal irrigation apparatus disclosed herein may be a reusable apparatus comprising a sterilisable container, a sterilisable spout, and a disposable nasal tip. After use, the nasal tip may be discarded and recycled while the container and spout are washed and then sterilized using a suitable method exemplified by boiling in water and/or washing in a hydrogen peroxide solution and/or washing in an alcohol solution (for example ethanol or isopropanol and the like). The container and spout should then be rinsed to remove traces of the hydrogen peroxide solution or washing in an alcohol solution.

It is to be noted that it is optional if so desired, for the open end of the resiliently compressible container to be provided with an internal female-threaded element and the proximal end of the spout to have a male-threaded component for demountable sealingly engagement with the internal female-threaded element of the open end of the resiliently compressible container. It is also optional, if so desired, for the distal end of the spout to have a female-threaded component, and for the proximal end of the nasal tip to have a male-threaded component for demountable sealingly engagement with the female-threaded element at the distal end of the spout.

Another embodiment of the present disclosure relates to nasal irrigation kits. The kits may be single-use kits or reusable kits. One example of a suitable kit comprises: (i) a resiliently compressible container pre-filled with a selected volume of a nasal irrigation solution and then sealed with a tamper-proof seal and a screw-on cap, (ii) a spout, and (iii) at least one nasal tip. A user would assemble the kit by removing the cap and the tamper-proof seal from the container, sealingly engage the female-threaded orifice of the spout with the male-threaded opening of the container, sealingly engage the nasal tip with the male-threaded distal end of the spout, after which, the nasal irrigation apparatus is ready for use. This embodiment of a kit may additionally comprise an extra bottle containing one or more single-use volumes of a selected nasal irrigation solution.

Another embodiment of a suitable kit may comprise: (i) an empty resiliently compressible container, (ii) a spout, and (iii) at least one nasal tip. A user would add a selected volume of a selected nasal irrigation solution, then sealingly engage the female-threaded orifice of the spout with the male-threaded opening of the container, sealingly engage the nasal tip with the male-threaded distal end of the spout, after which, the nasal irrigation apparatus is ready for use. This embodiment of a suitable kit may additionally comprise a tamper-proof sealed bottle containing one or more single-use volumes of a selected nasal irrigation solution.

The kits disclosed herein may comprise a spout with a shepherd's crook or alternatively, a straight linear spout, or alternatively may comprise both types of spouts. The kits may comprise one nasal tip or a plurality of nasal tips. The plurality of nasal tips may include one tip or multiple tips with a circular orifice, and/or one tip or multiple tips with a rectangular orifice, and/or one tip or multiple tips with a plurality of small-diameter misting orifices.

The nasal irrigation kits disclosed herein may comprise one or more different types of nasal irrigation solutions.

According to one embodiment, the kits may comprise a nasal irrigation solution for lavage of the nasopharynx and sinus cavities. Suitable irrigation solutions for nasal lavage are exemplified by isotonic saline solutions, isotonic phosphate-buffered solutions, Ringer's solution, and the like. A particularly suitable composition for nasal lavage is Ringer's solution. It is optional to add one or more ions to the Ringer's solution. Suitable ions include potassium (K⁺), rubidium (Rb⁺), magnesium (Mg²⁺), calcium (Ca²⁺), zinc (Zn²⁺), sulfate (SO₄²⁻), bromide (Br⁻), and the like.

According to another embodiment, the kits may comprise a nasal solution for delivery of an antibiotic to the nasopharynx and sinus cavities for nasal lavage by a user while they are experiencing cold or flu symptoms. A suitable nasal irrigation solution for delivery of an antibiotic may be based on Ringer's solution, alternatively isotonic saline solutions or isotonic phosphate-buffered solutions, to which has been added an antibiotic. Examples of suitable antibiotics include amikacin, amoxicillin, gentamicin, kanamycin, neomycin, netilmicin, paromomycin, tobramycin, geldanamycin, herbimycin, carbacephem (loracarbef), ertapenem, doripenem, imipenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, clarithromycin, clavulanic acid, clindamycin, colistimethate teicoplanin, azithromycin, dirithromycin, erythromycin, troleandomycin, telithromycin, aztreonam, ampicillin, azlocillin, bacampicillin, carbenicillin, cloxacillin, dicloxacillin, flucloxacillin, mezlocillin, meticillin, nafcillin, norfloxacin, oxacillin, penicillin G, penicillin V, piperacillin, pvampicillin, pivmecillinam, ticarcillin, bacitracin, colistin, colimycin, polymyxin B, ciprofloxacin, enoxacin, gatifloxacin, levofloxacin, lomefloxacin, moxifloxacin, ofloxacin, trovafloxacin, grepafloxacin, sparfloxacin, afenide, prontosil, sulfacetamide, metronidazole, sulfamethizole, sulfanilimide, sulfamethoxazole, sulfisoxazole, trimethoprim, trimethoprim-sulfamethoxazole, demeclocycline, doxycycline, oxytetracycline, tetracycline, arsphenamine, chloramphenicol, chlorhexidine, lincomycin, ethambutol, fosfomycin, furazolidone, isoniazid, linezolid, mupirocin, nitrofurantoin, platensimycin, pyrazinamide, quinupristin/dalfopristin, rifampin, thiamphenicol, rifampicin, minocycline, sultamicillin, sulbactam, sulphonamides, mitomycin, spectinomycin, spiramycin, roxithromycin, meropenem, azithromycin and sulfamethoxazole/trimethoprim, and the like. It is optional to add a vasoconstrictor to the present antibiotic nasal irrigation solutions. Examples of suitable vasoconstrictors include phenylephrine, pseudoephedrine, and the like. It is optional to add one or more ions to the present antibiotic nasal irrigation solutions. Suitable ions include K⁺, Rb⁺, Mg²⁺, Ca²⁺, Zn²⁺, SO₄²⁻, Br, and the like.

According to another embodiment, the kits may comprise a nasal solution for delivery of an antihistamine and/or a steroid to the nasopharynx and sinus cavities for nasal lavage by a user while they are experiencing allergy symptoms. A suitable nasal irrigation solution for delivery of an antibiotic may be based on Ringer's solution, alternatively isotonic saline solutions or isotonic phosphate-buffered solutions, to which has been added an antibiotic. Examples of suitable antihistamines include Hi-antihistamines. Suitable Hi-antihistamines include acrivastine, azelastine, bilastine, brompheniramine, buclizine, bromodiphenhydramine, carbinoxamine, chlorpromazine, cyclizine, chlorphenamine, chlorodiphenhydramine, clemastine, cyproheptadine, dexbrompheniramine, dexchlorpheniramine, dimenhydrinate, dimetindene, diphenhydramine, doxylamine, ebastine, embramine, fexofenadine, hydroxyzine, loratadine, meclozine, mirtazapine, olopatadine, orphenadrine, phenindamine, pheniramine, phenyltoloxamine, promethazine, quetiapine, rupatadine, tripelennamine, triprolidine, and the like. Examples of suitable steroids include triamcinolone and its derivatives (particularly the diacetate, hexacetonide, and acetonide), betamethasone and its derivatives (including particularly the dipropionate, benzoate, sodium phosphate, acetate, and valerate), dexamethasone and its derivatives (particularly the dipropionate and valerate), flunisolide, prednisone and its derivatives (particularly its acetate), prednisolone and its derivatives (particularly its acetate, sodium phosphate and tebutate), methylprednisolone and its derivatives (particularly its acetate and sodium succinate), fluocinolone and its derivatives (particularly the acetonide), diflorasone and its derivatives (particularly the diacetate), halcinonide, desoximetasone (desoxymethasone), diflucortolone and its derivatives (particularly the valerate), flucloronide (fluclorolone acetonide), fluocinonide, fluocortolone, fluprednidene and its derivatives (particularly the acetate), flurandrenolide (flurandrenolone), clobetasol and its derivatives (particularly the propionate), clobetasone and its derivatives (particularly the butyrate), alclometasone, flumethasone and its derivatives (particularly the pivalate), fluocortolone and its derivatives (particularly the hexanoate), amcinonide, beclometasone and its derivatives (particularly the dipropionate), fluticasone and its derivatives (particularly the propionate), difluprednate, prednicarbate, flurandrenolide, mometasone and desonide. It is optional to add a vasoconstrictor to the present allergy-relief nasal irrigation solutions. Examples of suitable vasoconstrictors include phenylephrine, pseudoephedrine, and the like. It is optional to add one or more ions to the present allergy-relief nasal irrigation solutions. Suitable ions include K⁺, Rb⁺, Mg²⁺, Ca²⁺, Zn²⁺, SO₄²⁻, Br, and the like.

According to another embodiment, the kits may comprise a nasal irrigation solution for lavage of the nasopharynx and sinus cavities for the purpose of soothing irritated mucous membranes, for example, after periods of nasal inflammation or after allergy attacks or after a cold or flu. A suitable nasal irrigation solution for soothing irritated mucosal membranes may be based on Ringer's solution, alternatively isotonic saline solutions or isotonic phosphate-buffered solutions, to which has been added an emollient. Examples of suitable emollients include glycerol, mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclome hicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soyabean, octyl palmitate, caprylic/capric triglyceride, glyceryl cocoate, and the like. It is optional to add to such nasal soothing solutions, an antioxidant. Examples of suitable antioxidants include a citric acid, butylated hydroxytoluene (BHT), ascorbic acid, glutathione, retinol, a-tocopherol, β-carotene, a-carotene, ubiquinone, butylated hydroxyanisole, ethyl enediaminetetraacetic acid, selenium, zinc, lignan, uric acid, lipoic acid, N-acetylcysteine, and the like. It is optional to add a vasoconstrictor to the present nasal soothing irrigation solutions. Examples of suitable vasoconstrictors include phenylephrine, pseudoephedrine, and the like. It is optional to add one or more ions to the present nasal soothing irrigation solutions. Suitable ions include K⁺, Rb⁺, Mg²⁺, Ca²⁺, Zn²⁺, SO₄²⁻, Br, and the like.

1. An apparatus for delivering a single-use volume of a nasal irrigation solution, comprising:
a resiliently compressible cylindrical container having a curvilinear body, said container having at least one pair of opposed channels molded thereinto; and
a spout;
wherein the container has a threaded open end, and the spout has a threaded proximal end for demountable sealingly engagement with the threaded open end of the container,

2. The apparatus according to claim 1, wherein the pair of opposed channels molded into the container extend around the curvilinear body from the range of about 15% to about 50% of a circumference of the curvilinear body.

3. The apparatus according to claim 1, wherein the curvilinear body of the cylindrical container has a round shape or an elliptical shape or an asymmetrical shape.

4. The apparatus according to claim 1, wherein the spout is formed in a shape of a shepherd's crook.

5. The apparatus according to claim 1, wherein the spout has a straight linear shape.

6. The apparatus according to claim 1, wherein the nasal tip is integrally molded with the spout.

7. The apparatus according to claim 1, wherein the spout has a threaded distal end for demountable sealingly engagement with a threaded proximal end of a nasal tip.

8. The apparatus according to claim 7, wherein the nasal tip has a distal end with one of a circular orifice, a rectangular orifice, and a plurality of small-diameter misting orifices.

9. A nasal irrigation kit comprising the apparatus of claim 1.

10. The nasal irrigation kit according to claim 9, comprising a first spout with a shape of a shepherd's crook and a second spout with a straight linear shape.

11. The nasal irrigation kit according to claim 9, additionally comprising one or more nasal tips.

12. The nasal irrigation kit according to claim 9, comprising a spout with an integrally molded nasal tip.

13. The nasal irrigation kit according to claim 9, wherein the resiliently compressible container has been filled with a volume of a nasal irrigation solution, said filled container sealed with a tamper-proof seal.

14. The nasal irrigation kit according to claim 9, additionally comprising a bottle filled with a volume of a nasal irrigation solution, said bottle sealed with a tamper-proof seal.

15. The nasal irrigation kit of claim 9, comprising one or more containers of one of a nasal irrigation solution, an antibiotic-containing nasal irrigation solution, an antihistamine-containing nasal irrigation solution, a steroid-containing nasal irrigation solution, an emollient-containing solution, and an antioxidant-containing nasal irrigation solution.

16. The nasal irrigation kit of claim 15, wherein the nasal irrigation solution additionally contains a vasoconstrictor.

17. The nasal irrigation kit of claim 15, wherein the nasal irrigation solution additionally contains one or more ions selected from the group consisting of potassium, rubidium, magnesium, calcium, zinc, sulfate, and bromide.
